(19) United States
(12) Patent Application Publication
(10) Pub. No.: US 2026/0097208 A1
(43) Pub. Date: Apr. 9, 2026

(54) PORTABLE VAGUS-NERVE ELECTROSTIMULATION DEVICE SYNCHRONISED WITH THE BREATHING CYCLE

(71) Applicant: FUNDACION CARDIOVASCULAR DE COLOMBIA, Santander (CO)

(72) Inventors: Edwin Orlando Gonzalez Serrano, Santander (CO); Gina Constanza Riascos Chalapud, Santander (CO); Elkin David Diaz Plata, Santander (CO); Sandra Milena Sanabria Barrera, Santander (CO); Cristian Jesus Gomez Vecino, Santander (CO); Maria Daniela Rios Duran, Santander (CO)

(21) Appl. No.: 19/113,941

(22) PCT Filed: Sep. 22, 2023
(86) PCT No.: PCT/IB2023/059407

§ 371 Date: Mar. 21, 2025

(30) Foreign Application Priority Data
Oct. 21, 2022 (CO) NC2022/0015061

(51) Int. Cl. A61N 1/36; A61N 1/02; A61N 1/04

(52) U.S. Cl. CPC A61N 1/36034; A61N 1/025; A61N 1/0456; A61N 1/0472; A61N 1/36025; A61N 1/36031; A61N 1/36036

(57) ABSTRACT

The invention is a non-implantable device electro stimulator Vagus nerve synchronized by thoracic movements in humans to improve the body motor function of patients after suffering an ischemic stroke, consisting of an atrial mechanism with four Electrodes that stimulate the nerve branches located in an earpiece that electrically stimulate the endings of the Vagus nerve that are located in the area of the Cavum Conchae and the Cymba Conchae of the left ear and the right ear and which in turn connects wirelessly to a mobile application that configures the stimulation parameters, also has a breathing cycle sensing band to sense the breathing placed around the user's chest that measures the expansion and recoil of the chest during breathing.

### TECHNOLOGY SECTOR

The invention is a non-implantable device develop to produce electrostimulation for the Vagus nerve which is synchronized by the respiratory cycle in humans, this device has been develop to get some multiple benefits in human disease like, movement recovery for the upper limbs in the body, this, after an ischemic stroke; this device could also serve for the treatment of migraine, major depression, among other medical conditions, consisting of an atrial mechanism, generating electrical stimulus generation device with four electrodes that stimulate the nerve branches that are in the Cymba Conchae and Cavum Conchae areas of the left or right ear; with two Electrodes located in an earpiece that electrically stimulate the Vagus nerve endings in the Cavum Conchae and in the Cymba Conchae of the left ear, and potentially with two electrodes located in the adapted earpiece that electrically couple to the Vagus nerve endings in the Cavum Conchae and in the Cymba Conchae of the right ear; a respiratory cycle sensing band to acquire the respiratory movement signal, placed around the user's chest that measures the expansion and retreat of the chest during breathing, which has a wireless connection with the atrial mechanism to synchronize electrostimulation with the respiratory cycle; and a smartphone app that configures the earpiece mechanism containing the Electrodes.

### STATE OF THE ART

For the treatment of pain and migraine by electrostimulation are known as the closest state of the art the Colombian patent application NC2016/0004437 “VAGUS NERVE ELECTROSTIMULATION DEVICE SYNCHRONIZED WITH THE RESPIRATORY CYCLE” which proposes a device with connecting elements for the synchronization of the emission of electrostimulation of the Vagus nerve with the connection of an electrocardiogram (ECG), Through the synchronization of the user's respiratory cycle, measuring breathing with Electrodes connected to the electrocardiogram (ECG). This antecedent proposes a solution that requires a wired Electrode that goes to the patient's ear transcutaneously for the treatment of pain. In contrast, this invention proposes a portable auricular device with external Electrodes that are electronically coupled with the nerve branches in the Cymba Conchae and Cavum Conchae of the Vagus nerve without requiring wired connections for the patient user away from the known solutions where electrostimulation of the Vagus nerve is determined by wired devices. Additionally, it does not have a connection to an electrocardiogram (ECG) but proposes a breathing cycle sensing band with wireless connection for the synchronization of the stimulation given by the atrial mechanism of generation of electrical stimulus, which, in turn, is configured by the application on a mobile device.

The invention is known the patent antecedent US2012035680A1 “Systems and Methods for Respiratory-Gated Auricular Vagal Afferent Nerve Stimulation” that requires identification and connection with two afferent nerves to the Vagus nerve with independent adjustments to make the connection, which is complemented by the method of patent US2020139126A1 “Systems and methods for respiratory-gated nerve stimulation” whose execution consists of determining a pulmonary characteristic of the user; and send the stimulation signal to the stimulation circuit to effect the user's electrical stimulation based on the user's lung characteristic. Unlike the new application, the portable Vagus nerve electrostimulation device synchronized with the respiratory cycle has a wireless data flow, wireless charging system and an atrial mechanism for generating electrical stimulus generation device for synchronization that could have four Electrodes that stimulate the nerve branches found in the Cymba and Cavum Conchae areas of the left ear and with possibilities in the right ear; With two Electrodes located in an earpiece that electrically attach to the Vagus nerve endings in the Cavum Conchae and in the Cymba conchae of the left ear and potentially with two Electrodes located in the adapted earpiece that electrically stimulate the Vagus nerve endings in the Cavum Conchae and in the Cymba conchae of the right ear.

The new invention contemplates a mechanism with sensor on the user's chest that is responsible for sensing the movement of the respiratory cycle (inspiration and expiration) of the user, indicating to the auricular mechanism when to provide the stimulus, according to the configurations made from a mobile application linked with the auricular mechanism of generation of electrical stimulus, novel feature that does not exist in any of the antecedents; since in the background it is not possible the portability or the displacement of the user. The criteria for solving the antecedents address one of between hypertension, inflammatory disorder, cardiovascular disease, chronic pain, anxiety disorder, disorder associated with chronic hypoxia/hypercapnia, primary disorder of the autonomic system or gastrointestinal disorder because they pretend to have contact with afferent nerves to the Vagus nerve depending on the identification of the branching unlike the new invention that proposes a unique coupling mechanism with four Electrodes to Cavum Conchae and in the Cymba conchae of the left ear and the right ear to achieve a therapeutic action in the functional sequelae of the cerebrovascular accident-Stroke, likewise, as in migraine and major depression.

### DESCRIPTION OF THE INVENTION

The invention is a non-implantable device electro stimulator of the Vagus nerve synchronized by thoracic movements of the respiratory cycle in humans to achieve a therapeutic action in the functional sequelae of stroke-stroke, likewise, such as migraine, major depression, hypertension, inflammatory disorder, cardiovascular disease, chronic pain, anxiety disorder, disorder associated with chronic hypoxia/hypercapnia, primary disorder of the autonomic system or gastrointestinal disorder, consisting of an atrial mechanism for generating electrical stimulation that could have four Electrodes that stimulate the nerve branches found in the Cymba and Cavum Conchae areas of the left ear and with possibilities of use in the right ear; with two Electrodes located in an earpiece that electrically stimulate the Vagus nerve endings in the Cavum Conchae and in the Cymba conchae of the left ear and potentially with two Electrodes located in the adapted earpiece that electrically stimulate the Vagus nerve endings in the Cavum Conchae and in the Cymba conchae of the right ear, a respiratory cycle sensing band to acquire the respiratory movement signal, placed around the user's chest that measures the expansion and retreat of the chest during breathing, which has a wireless connection with the atrial mechanism to synchronize electrostimulation with the respiratory cycle; and a mobile application that configures the auricular mechanism that contains the Electrodes.

### BRIEF DESCRIPTION OF FIGURES:

FIG. 1 shows the atrial mechanism's components that generates electrical stimulus generation device of the Vagus nerve synchronized with the respiratory cycle and the sensing band of the respiratory cycle that connects wirelessly to the atrial mechanism of generating electrical stimulus.

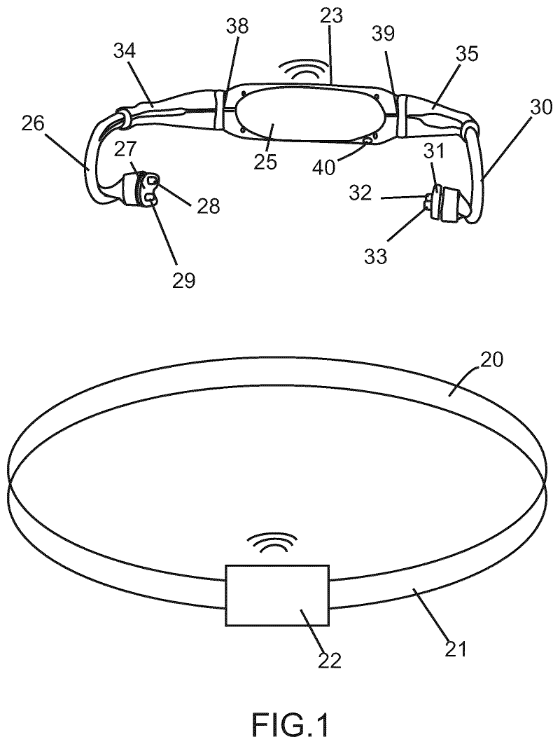

FIG. 2 shows a side view of the headphones of the atrial mechanism of generating electrical stimulus generation device of the Vagus nerve synchronized with the respiratory cycle.

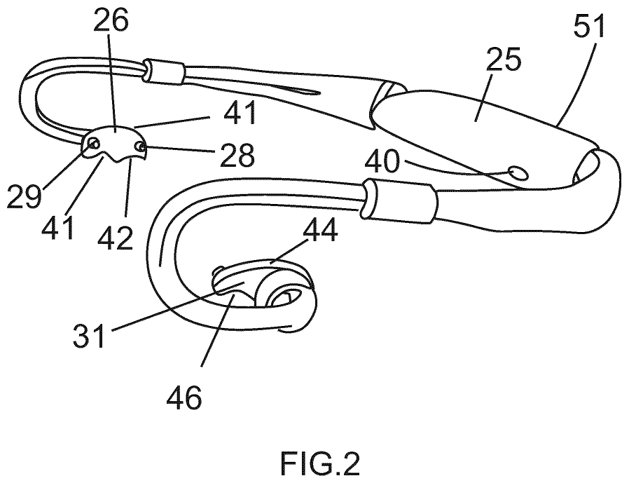

FIG. 3 shows a posterior perspective of the headphones and the housing of the atrial mechanism of generating electrical stimulus generation device of the Vagus nerve synchronized with the respiratory cycle.

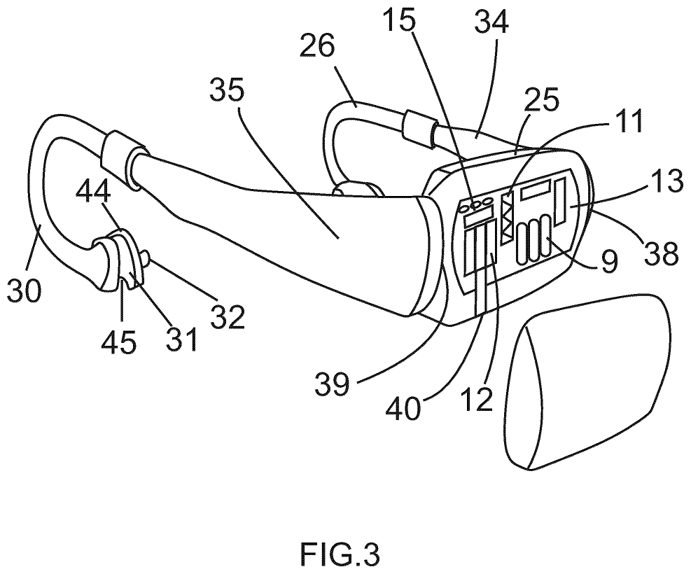

FIG. 4 shows a schematic of the housing and wireless charging coil of the respiratory cycle sensing band that connects wirelessly to the atrial electrical stimulus generation mechanism, adapted to receive the sensing signal, and generate an output signal.

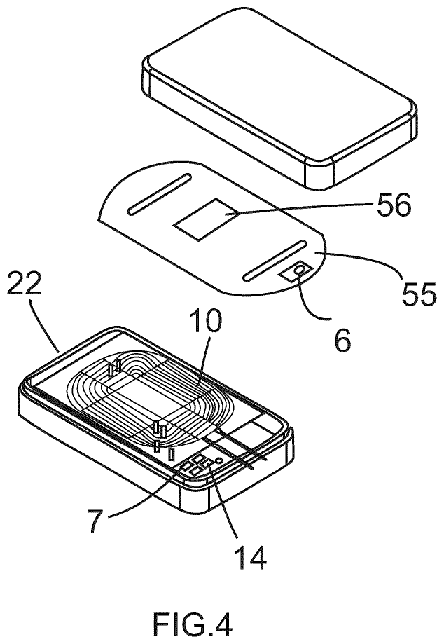

FIG. 5 shows the respiratory cycle sensing band around the user's chest, where the respiratory cycle sensing band allows synchronization of the respiratory cycle by the respiratory movements of the patient's chest.

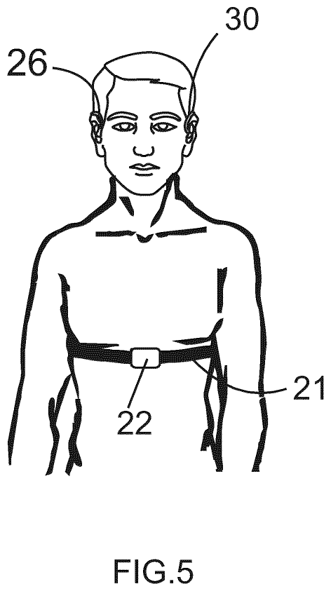

FIG. 6 shows the atrial mechanism of generating electrical stimulation of the Vagus nerve synchronized with the respiratory cycle of the user placed in a patient.

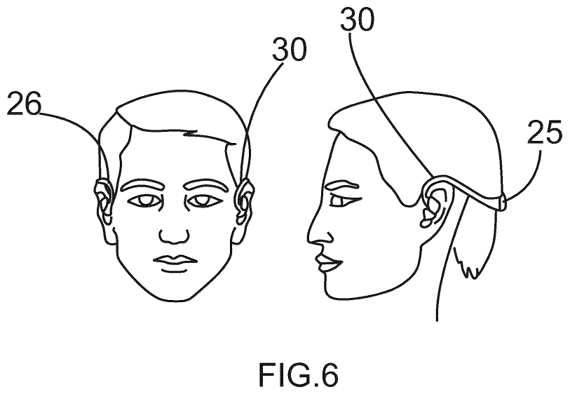

FIG. 7 shows the atrial mechanism of generating electrical stimulation of the Vagus nerve synchronized with the user's respiratory cycle with a spiral section path.

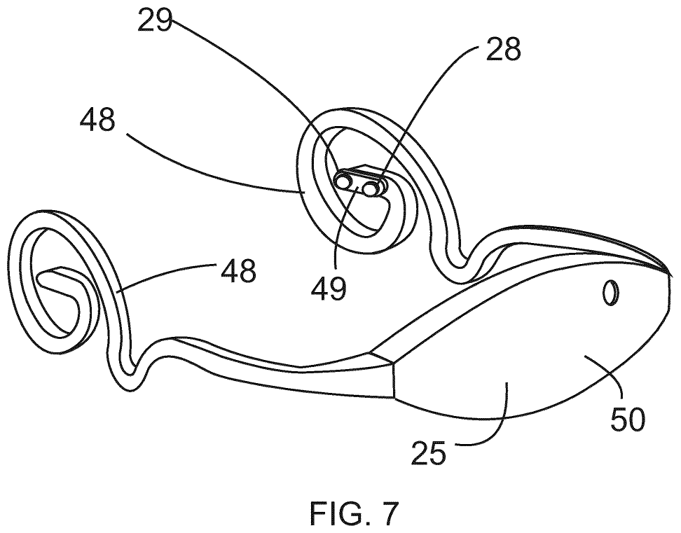

FIG. 8 shows the atrial mechanism of generating electrical stimulus generation device from the Vagus nerve synchronized with the user's respiratory cycle on their wireless charger.

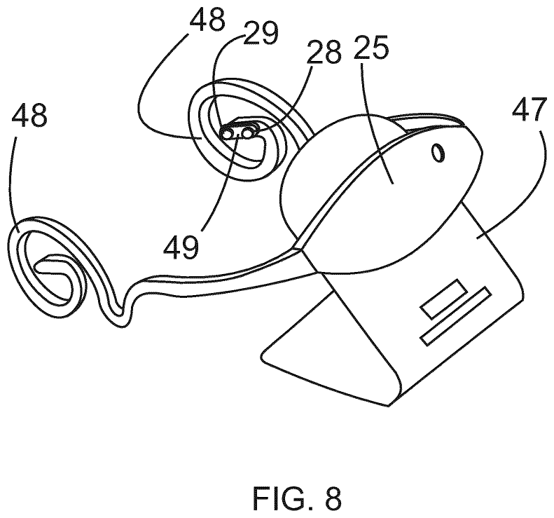

FIG. 9 shows in detail the support of an Electrode array, with a Cymba conchae Electrode and a Cavum conchae Electrode.

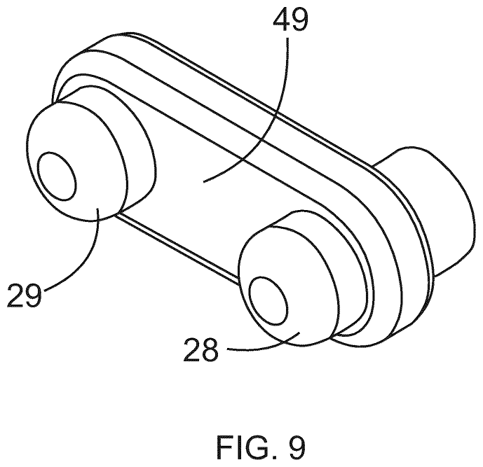

FIG. 10 shows a front view of the Vagus nerve electrical stimulus generating atrial mechanism card synchronized with the user's respiratory cycle.

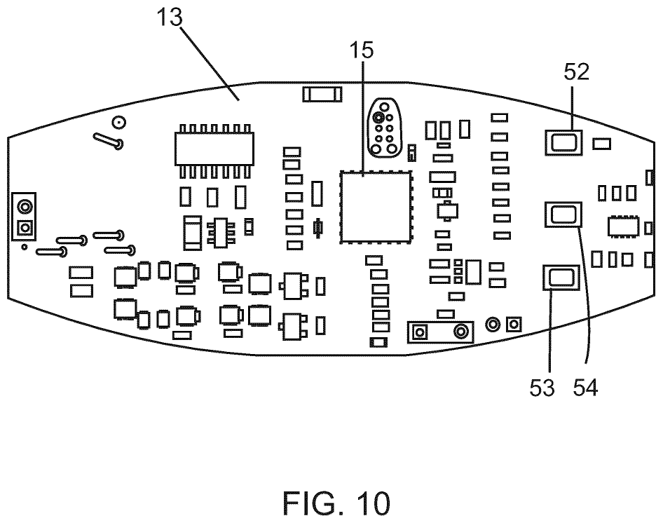

FIG. 11 shows a rear view of the Vagus nerve's electrical stimulus generation device in atrial mechanism card synchronized with the user's respiratory cycle.

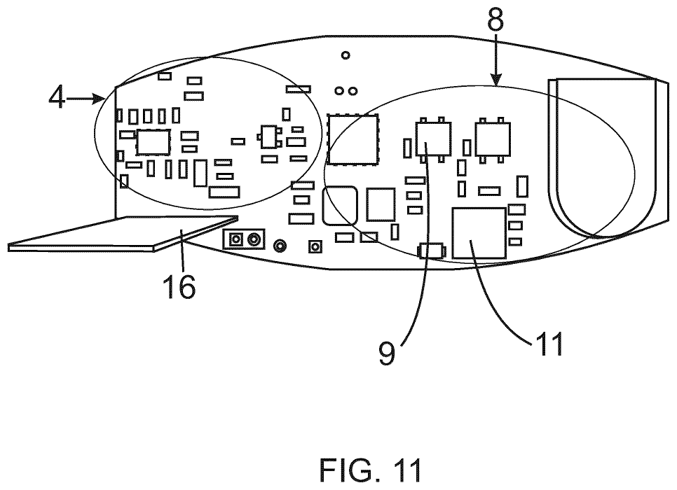

FIG. 12 shows a front view of the user's respiratory cycle sensing band card.

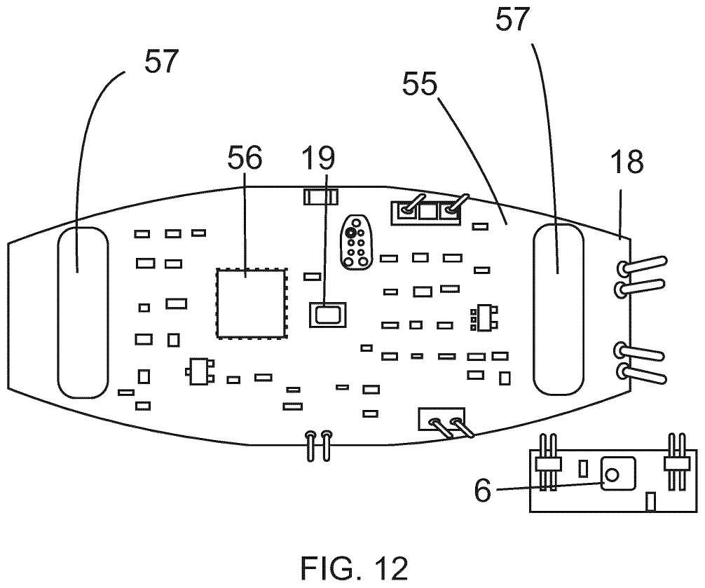

FIG. 13 shows a rear view of the user's respiratory cycle sensing band card.

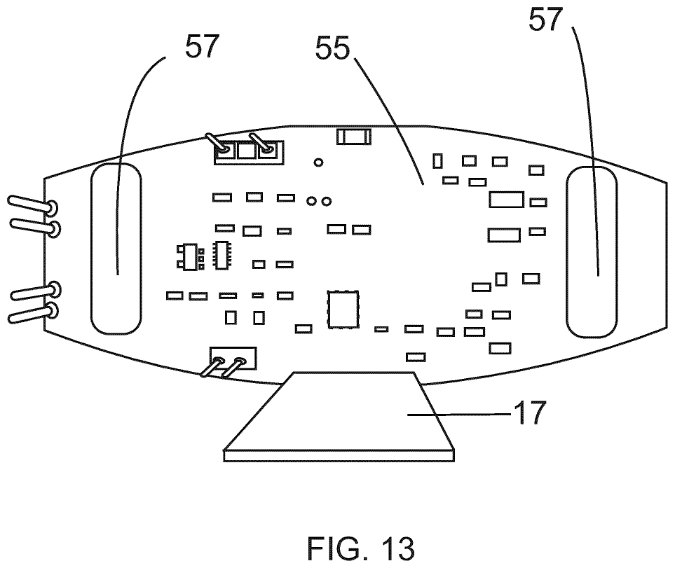

### TECHNICAL DESCRIPTION AND BEST WAY TO EXECUTE THE INVENTION

The portable device of electrostimulation of the Vagus nerve synchronized with the respiratory cycle favors the rehabilitation process in patients with cerebrovascular accidents-stroke with a solution of components that interact wirelessly with each other and allow the mobility of the user, its displacement and attachable to the human physiognomy that can be used as a garment. This device could also be used to achieve therapeutic action in migraine, major depression, hypertension, inflammatory disorder, cardiovascular disease, chronic pain, anxiety disorder, disorder associated with chronic hypoxia/hypercapnia, primary autonomic system disorder or gastrointestinal disorder.

The portable device of electrostimulation of the Vagus nerve synchronized with breathing is made up of:

An atrial mechanism for generating electrical stimulus, responsible for providing the electrical stimulus generation device in the pinna, with an electronic battery and wireless charging module, a wireless and power communication module to generate the stimulus and intensity control module.

A respiratory cycle sensing band, also consisting of hardware and firmware, will manage sensing the patient's respiratory cycle to show to the auricular device when to provide the stimulus, according to the configurations made from a mobile application. It also has a battery, wireless communication, processing and has an ergonomic shape so as not to affect the comfort of the patient who requires its use.

A mobile configuration application that consists of a software development where the configuration of the headset and synchronization hardware is performed. The mobile application meets usability, scalability, and medical standards requirements to be easily used by medical personnel. From the mobile application it is possible to configure the parameters of the electrical stimulus generation device signal such as: signal frequency, pulse width, current amplitude, and stimulus time, additionally it is possible to set up the therapy time and the activation of synchronization with the respiratory cycle. The mobile application could have preset configurations for different pathologies. The mobile app interface is designed to display relevant information such as therapy time, treatment intensity, and patient sensitivity level.

The device has different characteristics of use, shape, function, production, structure, and image that allow to compose a device focused on the user, where the shape is attractive and harmonious with rounded shapes on its edges and vertices allowing manipulation without affecting the user's skin, the patient can also interact with the different controls without affecting the user's comfort. It is also highlighted design that allows to the user an easily way to find the different components of the mechanism such as pushbuttons and loading mechanisms. The learning time of the device is very short since the visual elements used allow a very intuitive use, also textures and materials are raised that facilitate the grip generating greater comfort, these materials have also been focused for easy and quick cleaning avoiding foci of bacteria that may affect the user.

The power supply mechanism has sufficient autonomy for the execution of the electrical stimulus generation device therapy thanks to the integrated batteries, which are recharged by wireless technology; It has electronic elements such as regulators (9) and voltage boosters (11), passive elements that through an interconnection configuration charge as an energy backup of the device, in addition to a wireless charging mechanism that consists of a base, with coherent and attachable design, which supplies energy wirelessly to the auricular mechanism of generation of electrical stimulus generation device and to the sensing band of the respiratory cycle.

The respiratory cycle sensing module of the breathing cycle sensing band consists of OEM cards, which consist of an HW and a known data sending communication protocol. Its operation has been designed to measure the physiological variable by sensors, adapt, and process the signals internally, to later send them under a specific coding, known as communication protocol, through Bluetooth wireless technology. The sensing module of the respiratory cycle has cards that through a force sensor gets the signal generated by the opening of the patient's chest, which will later be processed for the identification of the maximum and minimum of the same, thus generating the activation points of the electrical stimulation.

The atrial mechanism of electrical stimulus generator (23) is based on the impulse of transcutaneous electrostimulation starts the electronic circuit that supplies an electrical power not harmful to humans as showed by microcontroller (15). The electronic elements that make it up are represented by passive components such as resistors, capacitors, coils in conjunction with active elements such as amplifiers operations and high voltage elevators, which transform electrical impulses so that they are perceived by the human organism. Additionally, The atrial mechanism of electrical stimulus generator (23) has two buttons to control the current intensity by the patient without having to interrupt their therapy time.

The atrial mechanism of electrical stimulus generator (23) has a protective housing that is a structure that isolates its internal circuits with the outside, conformed by support structures to there locate components or electronic cards in a compact way, which are protected by the outer casing, in which its shape influences in a shocking way, color, design, as well as its labels, elements or interface.

The atrial mechanism of electrical stimulus generator (23) is configured to stimulate the atrial branch of the Vagus nerve (ABVN) on the skin surface of the user's ear; where the atrial mechanism of electrical stimulus generator (23) comprises at least four Electrodes stimulating the nerve branches in the Cymba Conchae and Cavum Conchae areas of the ears; with two Electrodes located in an earpiece that electrically stimulates the Vagus nerve endings in the Cavum Conchae and in the Cymba conchae of the left ear and potentially with two Electrodes located in the adapted earpiece that electrically stimulate the Vagus nerve endings in the Cavum Conchae and in the Cymba conchae of the right ear that synchronize with the respiratory cycle sensing band (21) of a respiratory cycle sensing module (22) consisting of OEM cards with an HW and a known communication protocol for sending data wirelessly to the atrial mechanism for generating The atrial mechanism of electrical stimulus generator (23) which in turn communicates wirelessly with a mobile application. The atrial mechanism of electrical stimulus generator (23) is located on the back of the skull inside a housing (25), whether it is a fixed housing (50) or a tilting housing (51), which is attached to the arms (34), (35) of two earpieces (26), (30); wherein the right earpiece (26) at its termination has attached a right plate (27) with a path of oval section (41) or spiral section (48), and two concavities (42), (43) which could rotate in its center or have fixed sections (49) housing a right rear Cymba conchae Electrode (28) and a right front Cavum Conchae Electrode (29), and wherein the left earpiece (30) at its termination has attached a left plate (31) with a path of oval section (44) or spiral section (48), and two concavities (45), (46) that could rotate in its center or have fixed sections (49) that house a left posterior Cymba Conchae Electrode (32) and a left frontal Cavum Conchae Electrode (33).

The portable Vagus nerve electrostimulation device synchronized with the respiratory cycle has Electrodes with biocompatible metals, non-toxic metals, where the design of the housing is made of a biocompatible material and preferably thermoplastic achieving a lightweight device.

This device has a design that contemplates the average anthropometric measurements to fit the size of the head; The mold containing the Electrodes contemplates a unique design that optimizes contact with the endings of the Vagus nerve in the Cavum Conchae and in the Cymba conchae, of the left ear and the right ear of the user.

The result of the design of the wireless headset meets aspects of ergonomics and comfort, light weight, perfect the contact of the Electrodes with the skin of the ear, ensures tightness through snap closures and the implementation of tapped buttons.

The housing (25) on its right side (38) attaches to a right arm (34) and on its left side (39) attaches to a left arm (35), allowing it to be placed on the back of the user's skull. This housing (25) houses a wireless power supply mechanism (40) that is coupled to a wireless base (47) and houses The atrial mechanism of electrical stimulus generator (23), consists about the stimulation circuit that is connected independently to the right rear Cymba conchae Electrode (28), to the right front Cavum Conchae Electrode (29), to the left posterior Cymba Conchae Electrode (32) and to the left front Cavum Conchae Electrode (33).

Inside the housing (25) is the electronic card (13) that has passive elements such as resistors, capacitors and coils that through an interconnection configuration allow the charging and unloading of the energy backup mechanism (12) of the device; On the front there are three pushbuttons, first button (52) to fulfill the functions of on and off, a second button (53) to increase the stimulus current and a third push button (54) to decrease the stimulus current. On the back of the electronic board (13) are the electronic elements such as regulators (9) and voltage boosters (11) that make up the elevator/regulator mechanism (8); and the wireless charging mechanism (4) with its wireless charging coil (16) that attaches parallel to the electronic card (13)

The device's components connection is done via BLE (Bluetooth Low Energy), which sets up the communication between The atrial mechanism of electrical stimulus generator (23) device and the mobile application on a smartphone, in the same way as the communication between the atrial mechanism of generating electrical stimulus generation device and the breathing sensing band. The atrial mechanism of electrical stimulus generator (23) has a stimulation circuit connected to the microcontroller (15) which is adapted to receive the output signal from the detection device and generate an electrostimulation signal; A first Electrode adapted to electrically sense an afferent nerve fiber from an atrial branch of a patient's Vagus nerve, where the stimulation circuit is adapted to deliver the electrostimulation signal to the first Electrode for selective stimulation of the nerve fiber to achieve therapeutic function. The stimulation circuit is housed in an enclosure, and the enclosure is sized and configured to be supported by the patient's two atria.

The stimulation circuit is coupled to the microcontroller which in turn is configured wirelessly through a mobile application. Electrical stimulation includes at least the following: Current pulse: continuous; Burst of biphasic square wave pulses, Frequency: 1-120 Hz; Current intensity: 1 mA to 10 mA; Pulse width: 100-500 microseconds; Stimulation duration: 0.5-2 s and Therapy time: 1-60 minutes.

It has an energy measurement circuit that watches the output of the stimulation circuit and feeds back to the microcontroller to keep configured the stimulation energy and has a wireless power supply mechanism (40) with QI standard which manages enabling the battery to be charged without the need for physical ports.

The portable Vagus nerve electrostimulation device synchronized with the respiratory cycle has a respiratory cycle sensing band (21) supplemented by a housing. This respiratory cycle sensing band (21) is a chest band that allows the reading of respiratory cycles by opening the patient's chest and is found around the patient's chest to send information as a detection signal. It has a microcontroller (56) connected by a wireless communication to the atrial mechanism of electrical stimulus generator (23); where the respiratory cycle sensing band (21) is configured to measure the expansion and retreat of the subject's chest during breathing.

The result of the design of the band meets ergonomics aspects and comfort, since it has the possibility of graduating by sizes, design of the housing is made of a biocompatible material and preferably thermoelastic achieving a lightweight device and enabling its disinfection with alcohol, ensures tightness through pressure closures. Finally, the design makes it unique due to its characteristic of harmonic aestheticism and minimalism with the wireless headset.

Inside the band is an electronic board (55) that communicates wirelessly via BLE (Bluetooth Low Energy) to the wireless device of The atrial mechanism of electrical stimulus generator (23); and is adapted to detect the thoracic movements of a patient's respiratory cycle and convert the detected muscle activity into a corresponding detection signal; has at least one microcontroller (56) configured to determine a respiratory cycle of the subject in the chest band; It is executed with at least one operation of inspiration and expiration and where the delivery of the stimulation signal includes timing a delivery of stimulations to the subject at the beginning of expiration or inspiration and extending through a portion of it.

The respiratory cycle sensing band (21) on his electronic board (55) has two side grooves (57) that are crossed by the strap extension (20) that surrounds the patient's chest when using the respiratory cycle sensing band (21). On the front of the electronic board (55) you can see the microcontroller (56), the on/off button (19), and the connectors (18) of the force sensor (6). On the back of the electronic board (55) the wireless charging mechanism is mainly configured with its wireless charging coil (17) that is coupled parallel to the electronic board (55).

The microcontroller (15) of the wireless to the atrial mechanism of electrical stimulus generator (23) is configured to finish delivering stimulations to the subject before the next cycle of inhalation or exhalation.

The microcontroller (15) of the wireless electrical stimulus generation device generation atrial mechanism is configured to receive a setting to figure out a synchronization of the pulmonary characteristic from a smartphone application.

The microcontroller (56) of the respiratory cycle sensing band is configured to communicate the maximums and/or minimums of muscle activity to the microcontroller (15) of the wireless to the atrial mechanism of electrical stimulus generator (23) to initiate the burst of electrostimulation pulses; This microcontroller (56) is configured to sense the subject's respiratory cycle by detecting the maximums and minimas of inspiration and expiration; and is also configured wirelessly to send the subject's detected muscle activity to the processor of the wireless to the atrial mechanism of electrical stimulus generator (23).

The respiratory cycle sensing band (21) has a wireless charging circuit with QI standard (14) which manages enabling the battery to be charged without the need for physical ports.

The mobile application is designed to perform the configuration of the parameters of the stimulation signal and communicates through BLE technology with auricular mechanism for generating wireless electrical stimulation. The mobile application has the possibility to pause, resume or conclude the therapy, additionally, the mobile application can manage the shutdown of the wireless electrical stimulus generation auricular mechanism, the breathing sensing band. View the status and battery charge level of the two devices. The application monitors and supplies the patient's specific traditional therapies to treat the medical conditions already mentioned, as well as having the possibility of having a record of metrics of defined variables to reflect an indicator of improvement status. (Frequency of migraine episodes, frequency of depression episodes, migraine pain indicator, use of smartphone camera to record recovery or improvement).

From the results of specific therapies (migraine, major depression, upper limb motor rehabilitation, hypertension, inflammatory disorder, cardiovascular disease, chronic pain, anxiety disorder, disorder associated with chronic hypoxia/hypercapnia, primary autonomic system disorder or gastrointestinal disorder), the application could create patient profiles to generate recommendations, identify patterns with potential to generate new ones. Complete therapy settings, generate predictions of medical conditions or complications by interoperability of other devices that measure other complementary physiological variables for therapies.

The electrical stimulation protocols execute the therapeutic function according to the treatment of the medical conditions to be treated with a placebo mode and an automatic synchronization mode with the respiratory cycles, which are configured with automatic stimulation packages according to data captured from the breath.

Method of operation (App interaction, Band, and wireless headset) with electrostimulation function including:

The atrial mechanism of generating electrical stimulus generation device receives an activation and could receive a signal of the respiratory cycle of the sensing band by a wireless means that detects the patient's muscle activity.

The characterization of the signal of the breathing cycle is conducted from the sensing band of the respiratory cycle who sends activation parameters to the atrial mechanism of generation of electrical stimulus generation device to start the burst of electrical pulses.

The atrial mechanism of generating electrical stimulus generation device senses and keeps the parameter settings set by the mobile application.

The entire data stream in the stimulation system is done by Bluetooth wireless communication.

The operation of the mobile application, it is characterized by:
a) Before stimulation:
Creation by the administrative staff of an account for medical staff.
Medical staff log into the mobile app and connect to the atrial mechanism of generating electrical stimulus generation device via BLE.
The application has registered predetermined parameters that can be changed by medical staff based on therapy and pain thresholds in the patient to electro stimulate.
Select whether the device will run synchronized to breathing or not.
b) During stimulation:
The mobile application shows the remaining time until the end of the electrostimulation session.
The mobile application displays the summary of the parameters used in the current session.
The mobile application allows you to pause, stop and continue electrostimulation.
c) After stimulation:
The mobile application stores the last registered parameter configuration to be used in a next session.
The mobile application allows the shutdown of the atrial mechanism for generating electrical stimulus generation device and the breathing sensing band.

This mobile application has among its functions:
The application shows by an icon the battery percentage of the earphone mechanism and the sensing band.
The app notifies when the respiratory cycle sensing band is connected.
The app alerts the user in case there is no connection with the devices.
The application allows to increase and decrease the current intensity.
The app notifies an alert in case the patient's respiratory rate exceeds the maximum. In this case, you should display a message if you want to stop or continue therapy.
The application allows you to reconfigure the parameters of the therapy in case any parameter is deconfigured.

1. Electronic transcutaneous Vagus nerve stimulator device synchronized by respiratory chest movements in humans to improve motor function of patients after suffering an ischemic stroke, and with potential for therapeutic use in patients with migraine, major depression, hypertension, inflammatory disorder, cardiovascular disease, chronic pain, anxiety disorder, disorder associated with chronic hypoxia/hypercapnia, primary autonomic system disorder or gastrointestinal disorder. This, with a wireless transcutaneous electrical nerve stimulator CHARACTERIZED by a respiratory cycle sensing band (21) with a respiratory cycle sensing module (22) consisting of OEM cards with an HW and a known communication protocol for sending data wirelessly to an atrial mechanism of electrical stimulus generator (23) is located on the back of the skull inside a housing (25), whether it is a fixed housing (50) or a tilting housing (51), which in turn communicates wirelessly with a mobile application and attaches to the arms (34), (35) of two earpieces (26), (30); wherein the right earpiece (26) at its termination has attached a right plate (27) with a path of oval section (41) or spiral section (48) and two concavities (42), (43) which are fixed or rotate in its center and which houses a right posterior Cymba conchae Electrode (28) and a right front Cavum Conchae Electrode (29), and wherein the left earpiece (30) at its termination has attached a left plate (31) with a trajectory is oval section (44) or spiral section (48) and two concavities (45), (46) that are fixed or rotate in its center and that houses a left posterior Cymba Conchae Electrode (32) and a left frontal Cavum Conchae Electrode (33).

2. Electronic transcutaneous electrical stimulator device of the Vagus nerve synchronized by respiratory thoracic movements in humans according to claim 1 WHEREIN the housing (25) has a transverse axis that on its right side (38) joins on its axis to the right arm (34) and on its left side (39) joins on its axis to the left arm (35).

3. Electronic device transcutaneous electrical stimulator of the Vagus nerve synchronized by respiratory thoracic movements in humans according to claim 1 WHEREIN the housing (25) houses a wireless power supply mechanism (40) with QI standard that is coupled in a wireless base (47) and houses the atrial mechanism of electrical stimulus generator (23), coupled to a microcontroller (15) configured wirelessly with a mobile application, whose electrical stimulation includes a direct current pulse with Burst of biphasic square wave pulses; Frequency: 1-120 Hz; Current intensity: 1 mA to 10 mA; Pulse width: 100-500 microseconds; Stimulation duration: 0.5-2 s and Therapy time: 1-60 minutes; and formed by the stimulation circuit that is connected to each Electrode, with connection to the right posterior Cymba Conchae Electrode (28), the right frontal Cavum Conchae Electrode (29), the left posterior Cymba Conchae Electrode (32) and the left frontal Cavum Conchae Electrode (33).

4. Electronic device transcutaneous electrical stimulator of the Vagus nerve synchronized by respiratory thoracic movements in humans according to claim 1 WHEREIN the atrial mechanism of electrical stimulus generator (23) inside its housing (25) has an electronic card (13) with an energy backup mechanism (12) of the device; whose electronic card (13) on the front has three pushbuttons, first push button (52) on and off, a second push button (53) for increasing the stimulus current and a third push button (54) for decreasing the stimulus current; and on the back of the electronic card (13) it has regulators (9) and voltage boosters (11) that make up the lift/regulator mechanism (8), and a wireless charging mechanism (4) with its wireless charging coil (16) that is coupled parallel to the electronic card (13).

5. Electronic transcutaneous electrical stimulator device of the Vagus nerve synchronized by respiratory thoracic movements in humans according to claim 1 WHEREIN the respiratory cycle sensing module (22) of the respiratory cycle sensing band (21) has an electronic board (55) with two side grooves (57) that are crossed by the strap extension (20) surrounding the patient's chest; current injectors (10); a wireless power supply mechanism with Ql standard (14); OEM cards (7) that send respiratory cycle information to the microcontroller (15) of the atrial mechanism of electrical stimulus generator (23) to activate the stimulation; whose front of the electronic board (55) has a microcontroller (56), an on/off button (19), and connectors (18) of a force sensor (6); and on the back of the electronic board (55) it has a wireless charging mechanism with its coil (17) that is coupled parallel to the electronic board (55).
